(19) United States
(12) Patent Application Publication
(10) Pub. No.: US 2026/0354705 A1
(43) Pub. Date: Nov. 12, 2026

(54) RESPIRATION RATE MEASUREMENT

(71) Applicant: Hoffmann-La Roche Inc., Little Falls, NJ (US)

(72) Inventors: Roman Peter KUSTER, Basel (CH); Thanner Malai PERUMAL, Oberwil, Basel-Landschaft (CH)

(21) Appl. No.: 19/118,336

(22) PCT Filed: Oct. 6, 2023
(86) PCT No.: PCT/EP2023/077720

§ 371 Date: Apr. 3, 2025

(30) Foreign Application Priority Data
Oct. 7, 2022 (EP) 22200393.1

(51) Int. Cl. A61B 5/08; A61B 5/00

(52) U.S. Cl. CPC A61B 5/0816; A61B 5/407; A61B 5/6898

(57) ABSTRACT

Respiration rate measurement A diagnostic device configured to measure a respiration rate of a user, the device comprising: at least one processor; a user interface; one or more sensors associated with the device; and a memory storing computer-readable instructions that, when executed by the at least one processor, cause the diagnostic device to: prompt, via the user interface, the user to provide a user input via the one or more sensors associated with the device each time the user is at a predetermined point during an inhalation cycle; receive a plurality of user inputs via the one or more sensors, each user input corresponding to a respective time at which the user is at a predetermined point during an inhalation cycle; generate, in response to receiving each user input, a timestamp associated with the respective user input; apply a respiration rate model to data comprising the plurality of generated time stamps, wherein the respiration rate model calculates a respiration rate of the user based on the generated time stamps; and output the calculated respiration rate.

### TECHNICAL FIELD OF THE INVENTION

The present invention relates to diagnostic devices and computer-implemented methods of measuring a subject's respiration rate.

### BACKGROUND TO THE INVENTION

Spinal Muscular Atrophy (SMA) is associated with severe breathing difficulties. People living with SMA (PlwSMA) typically show a rapid shallow breathing pattern dominated by the diaphragm {Bourke 2014, PMID 24532751}. Further, PlwSMA typically show an impaired ability to breathe slowly and deeply. The diaphragm domination is the consequence of having the chest muscles more affected by the muscle atrophy than the abdominal muscles. The thinning of the chest muscles can even lead to thoraco-abdominal asynchrony (with paradoxical breathing as the most severe form), in which the muscular contraction of the diaphragm is wasted to distort the chest wall rather than to inflate the lungs, making breathing very inefficient {LoMauro et al. 2014, PMID 24632504}. Chest muscle weakness might also result in inefficient coughing, bell-shaped chest anatomy, hypoventilation, or atelectasis (collapse or closure of part of the lungs). It is quite common for PlwSMA to use (pressure or volume controlled) non-invasive ventilators for part time of the day (referred to as non-permanent ventilation if <16 hours a day) {Mercuri et al. 2017, ISBN 9780128036853}. Non-invasive ventilators usually change the breathing pattern by reducing the amount of thoraco-abdominal asynchrony and usually define a minimal breathing rate below which they force the breathing {Lissoni et al. 1998, PMID 9635553}.

The most commonly used clinical measure to assess lung function in PlwSMA is forced vital capacity (FVC), typically expressed as % FVC predicted to account for age and sex differences {LoMauro et al. 2016, PMID 27820869}. A healthy person has a % FVC between 80 and 120 (100 is the average by definition), while PlwSMA could have a &FVC as low as 30 or even lower. The % FVC is the most important measure of respiratory function in clinical practice, but its actual relevance to daily life must be questioned, and differences in values <30 might be irrelevant. Another measure that is probably more relevant to everyday life is the peak cough flow (PCF), a measure that indicates the strength of coughing and thus the ability to clean the airways {Chatwin et al. 2018, PMID: 29501255}.

In order to diagnose and track the progress of many diseases, for example, spinal muscular atrophy (referred to herein as “SMA”), it is desirable to obtain a reliable value of the respiration rate of a user, in a manner which is non-invasive and can be performed e.g. at home by a subject.

### SUMMARY OF THE INVENTION

The present invention, at a high-level, provides a device and computer-implemented method of determining a respiration rate of a user. More specifically, a user is prompted to provide an input to the device each time they reach a predetermined point in an inhalation cycle. From a series of inputs, the device is then able to calculate a respiration rate.

Accordingly, a first aspect of the present invention provides a diagnostic device configured to measure a respiration rate of a user, the device comprising: at least one processor; one or more sensors associated with the device; a user interface; and a memory storing computer-readable instructions that, when executed by the at least one processor, cause the diagnostic device to: prompt, via the user interface, the user to provide a user input, via the one or more sensors associated with the device, each time the user is at a predetermined point during an inhalation cycle; receive a plurality of user inputs via the one or more sensors, each user input corresponding to a respective time at which the user is at a predetermined point during an inhalation cycle; generate, in response to receiving each user input, a timestamp associated with the respective user input; apply a respiration rate model to data comprising the plurality of generated time stamps, wherein the respiration rate model calculates a respiration rate of the user based on the generated time stamps; and output the calculated respiration rate.

The combination of the steps of: prompting the user; receiving the plurality of user inputs; generating the timestamps; applying the respiration rate model; and, outputting the calculated respiration rate may correspond to conducting a “respiration rate test”. That is, the computer-readable instructions, when executed by the at least one processor, may cause the device to conduct a respiration rate test, which may include the above-mentioned steps.

It is known that respiration rate often correlates with forced vital capacity and peak cough flow, both of which features can also be useful in making clinical assessments. By measuring respiration rate using a diagnostic device according to the first aspect of the present invention, it may be possible to track, effectively, the progress of various muscular disabilities such as SMA in a subject by active testing of the subject. As is described in detail later in this application, the diagnostic device according to the first aspect of the present invention may use the calculated respiration rate to indicate and/or track the presence or progression of a muscular disability, such as SMA, in a subject or user.

The computer-readable instructions, when executed by the at least one processor, may cause the diagnostic device to prompt the user via the user interface to breathe at an indicated breathing rate and/or breathing depth. The computer-readable instructions, when executed by the at least one processor, may cause the diagnostic device to conduct a plurality of respiration rate tests, wherein the user is prompted to breathe at a different breathing rate and/or depth in each respiration rate test. For example, the computer-readable instructions, when executed by the at least one processor, may cause the diagnostic device to prompt the user to breathe normally, to breathe slowly and deeply, and/or to breathe quickly and shallowly.

The breathing rate and/or depth may be indicated by a colour displayed on the user interface. For example, the colour green may prompt the user to breathe normally. Additionally, or alternatively, the breathing rate and/or depth may be indicated by text displayed on the user interface. For example, text displayed on the user interface may read “breathe normally”.

The computer-readable instructions, when executed by the at least one processor, may cause the diagnostic device to indicate to the user, for example via the user interface, the time duration over which the user should provide user inputs for a given respiration rate test. The indication may correspond to a countdown, for example. The time duration may be 10 seconds or more, or 30 seconds or less, for example 20 seconds.

In preferred implementations, the device is or comprises a smartphone. This is advantageous because smartphones are possessed by virtually everyone nowadays. By implementing a computer-implemented process such as the one described on a smartphone, a user need not attend e.g. a hospital or other clinical setting in order for the respiration rate to be measured. Other kinds of diagnostic device may be used, e.g. a tablet, a laptop computer, a desktop computer, or the like. Alternatively, the diagnostic device may be a dedicated respiration rate measuring device.

The diagnostic device preferably further comprises a display component which is configured to display the user interface. Preferably, the display component is in the form of a screen such as a touchscreen. In implementations in which the display component comprises a touchscreen, the touchscreen preferably includes the one or more sensors associated with the device. In those cases, the sensors may comprise resistive sensors, capacitive sensors, surface acoustic wave sensors, infrared grid sensors, infrared acrylic projection sensors, optical imaging sensors, piezoelectric sensors, and/or acoustic pulse recognition sensors. In most cases the one or more sensors are capacitive sensors, since these are most commonly used in smartphones. Capacitive sensors work on the basis that when a person touches the screen, its electrostatic field is distorted, which registers in a change in capacitance.

We now discuss in more detail the nature and operation of the respiration rate model, which is applied to the data comprising the plurality of timestamps in order to calculate the respiration rate. The respiration model may be configured to calculate a time difference between two time stamps of the plurality of timestamps, and to calculate the respiration rate based on a reciprocal of the calculated time difference. In some cases, the earlier of the two time stamps may immediately precede the later of the two time stamps. In other words, the two time stamps may be consecutive time stamps. Alternatively, there may be n time stamps between the earlier of the two time stamps and the later of the two time stamps, and the respiration rate model is configured to calculate the respiration rate by multiplying the reciprocal of the time difference by (n+1). Note that a time difference between two consecutive time stamps may correspond to a breath duration. In some examples, the respiration model may be configured to calculate a mean breath duration by summing a plurality of time differences, each time difference being the difference between two consecutive time stamps, and dividing the sum by n, where n is the number of time differences. The respiration rate model may then calculate a mean respiration rate by taking the inverse of the mean breath duration.

By inverting the time difference, or breath duration, as outlined above, the respiration rate model is able to calculate the respiration rate in units of breaths per second. In some cases, the respiration rate model may be further configured to multiple the inverted time difference or breath duration (i.e. the reciprocal) by sixty in order to obtain the respiration rate in units of breaths per minute.

We now discuss how the calculated respiration rate may be used to indicate a presence or a progression of a muscular disability, such as SMA. The computer-readable instructions, when executed by the at least one processor, may cause the diagnostic device to apply a clinical interpretation model to the calculated respiration rate. The clinical interpretation model may output an indication of the presence or absence of a muscular disability, such as SMA, in the user, or an indication of the progression of a muscular disability in the user. Applying the clinical interpretation model may include applying the clinical interpretation model to two or more calculated respiration rates, each of the two or more calculated respiration rates calculated in a different respiration rate test. The clinical interpretation model may be configured to calculate a frequency ratio of the two or more calculated respiration rates. The indication of the presence or absence of the muscular disability may be determined based on the calculated frequency ratio. In some examples, the frequency ratio may correspond to a ratio of a second respiration rate, calculated in a second respiration rate test, to a first respiration rate, calculated in a first respiration rate test. The first respiration rate test may correspond to a test in which the user is prompted to breath normally. The second respiration rate test may correspond to a test in which the user is prompted to breath deeply and/or slowly. The first respiration rate test (the “normal breathing” test) may be conducted before the second respiration rate test (the “deep and/or slow breathing” test). Conducting the normal breathing test before conducting the deep and/or slow breathing test may mean the user is more likely to breath normally when prompted to breath normally, compared to if the tests were conducted the other way round.

The clinical interpretation model may be configured to compare the frequency ratio to a predetermined value, and, based on the comparison, to output an indication of the presence or absence of the muscular disability, such as SMA. In particular, the clinical interpretation model may be configured to determine whether the frequency ratio is greater than a predetermined threshold, and if it is determined that the frequency ratio is greater than the predetermined threshold, to output an indication of the presence of a muscular disability (e.g., that the user is a PlwSMA), and/or if it is determined that the frequency ratio is less than or equal to the predetermined threshold, to output an indication of the absence of the muscular disability. For example, the predetermined threshold may be at least 0.5 and no greater than 0.8. In some examples the predetermined threshold may be at least 0.65 and no greater than 0.75. In some examples the predetermined threshold may be at least 0.68 and no greater than 0.73. For example, the predetermined threshold may be approximately 0.7, for example 0.71. That is, a frequency ratio of >0.71 may indicate that a user is a PlwSMA, and a frequency ratio of <0.71 may indicate that a user is not a PlwSMA. PlwSMA may slow their breathing rate for slow and deep breathing by <29% compared to normal breathing, whereas healthy individuals may slow their breathing rate for slow and deep breathing by >29% compared to normal breathing.

The predetermined point during the inhalation cycle may be the start of the inhalation cycle, i.e. when the user begins to breathe in. This allows a more reliable respiration rate to be obtained, because a user is more easily able to identify the point at which they begin breathing.

A second aspect of the present invention provides a computer-implemented method of measuring respiration rate in a subject. The computer-implemented method comprises: prompting, via a user interface, the subject to provide an input, via the one or more sensors associated with the device, each time the subject is at a predetermined point during an inhalation cycle; receiving a plurality of user inputs via the one or more sensors, each user input corresponding to a respective time at which the user is at a predetermined point during an inhalation cycle; generating, in response to receiving each user input, a timestamp associated with the respective user input; applying a respiration rate model to the plurality of generated time stamps, wherein the respiration rate model is configured to calculate a respiration rate of the user based on the generated time stamps; and outputting the calculated respiration rate. In preferred cases, the computer-implemented method of the second aspect of the invention is executed by a processor of a diagnostic device such as the diagnostic device of the first aspect of the invention. It will be appreciated that the optional features set out above, in respect of the first aspect of the invention, apply equally well to the second aspect of the invention except where context clearly dictates otherwise, or whether such a combination of features is clearly technically incompatible. For example, the computer-implemented method may further comprise applying a clinical interpretation model to the calculated respiration rate. The clinical interpretation model may output an indication of the presence or absence of a muscular disability, such as SMA.

A third aspect of the invention provides a computer program comprising instructions which when executed by a processor of a computer (or other suitable data processing device) cause the processor to execute the computer-implemented method of the second aspect of the invention. A further aspect of the invention provides a computer-readable storage medium having stored thereon the computer program of the third aspect of the invention.

The invention includes the combination of the aspects and preferred features described except where such a combination is clearly impermissible or expressly avoided.

### BRIEF DESCRIPTION OF THE DRAWINGS

Embodiments of the present invention will now be described with reference to the accompanying drawings, in which:

FIG. 1 is a diagram of an example environment in which a diagnostic device for assessing respiration rate of a subject is provided.

FIG. 2 is a flow diagram of a computer-implemented method for assessing the respiration rate of a user.

FIG. 3 is a flow diagram of a computer-implemented method for determining an indication of the presence or absence of a muscular disability, such as SMA.

FIG. 4 is a plot showing frequency ratios calculated for PlwSMA and for healthy individuals.

FIG. 5 illustrates one example of a network architecture and data processing device that may be used to implement one or more illustrative aspects described herein.

### DETAILED DESCRIPTION OF THE DRAWINGS

Aspects and embodiments of the present invention will now be discussed with reference to the accompanying figures. Further aspects and embodiments will be apparent to those skilled in the art. All documents mentioned in this text are incorporated herein by reference.

In the following description of various aspects, reference is made to the accompanying drawings, which form a part hereof, and in which is shown by way of illustration various embodiments in which aspects described herein may be practiced. It is to be understood that other aspects and/or embodiments may be utilized, and structural and functional modifications may be made without departing from the scope of the described aspects and embodiments.

Aspects described herein are capable of other embodiments and of being practiced or being carried out in various ways. Also, it is to be understood that the phraseology and terminology used herein are for the purpose of description and should not be regarded as limiting. Rather, the phrases and terms used herein are to be given their broadest interpretation and meaning. The use of “including” and “comprising” and variations thereof is meant to encompass the items listed Thereafter and equivalents thereof as well as additional items and equivalents thereof. The use of the terms “mounted,” “connected,” “coupled,” “positioned,” “engaged” and similar terms, is meant to include both direct and indirect mounting, connecting, coupling, positioning and engaging.

Systems, methods and devices described herein provide a diagnostic device and computer-implemented methods for assessing, measuring, or determining the respiration rate of a patient, for example a patient suffering from a muscular disability, such as particular SMA. In some cases, the diagnostic device may be in the form of a mobile, in particular a smartphone, on which a particular software application is installed. The software application may be configured to execute (or cause the processor of the mobile device) the corresponding computer-implemented method.

In some cases, the diagnostic obtains or receives sensor data from one or more sensors associated with the mobile device as the subject interacts with the software application using the mobile device. In some cases, the sensors may be within the mobile device. In some cases, the respiration rate is derived, calculated, or extracted from the received or obtained sensor data. In some cases, the assessment of the symptom severity and progression of a muscular disability, in particular SMA, in the subject may be determined based on the extracted sensor features.

In implementations of the present invention, the diagnostic device may prompt the subject to perform a diagnostic tasks. In some cases, the diagnostic tasks are anchored in or modelled after established methods and standardized tests. In some cases, in response to the subject performing the diagnostic task, the diagnostic obtains or receives sensor data via one or more sensors. In some cases, the sensors may be within a mobile device or wearable sensors worn by the subject. In some cases, sensor features associated with the symptoms of a muscular disability, in particular SMA, are extracted from the received or obtained sensor data. In some cases, the assessment of the symptom severity and progression of a muscular disability, in particular SMA, in the subject is determined based on the extracted features of the sensor data.

Assessments of symptom severity and progression of a muscular disability, in particular SMA, using diagnostics according to the present disclosure correlate sufficiently with the assessments based on clinical results and may thus replace clinical subject monitoring and testing. Example diagnostics according to the present disclosure may be used in an out of clinic environment, and therefore have advantages in cost, ease of subject monitoring and convenience to the subject. This facilitates frequent, in particular daily, subject monitoring and testing, resulting in a better understanding of the disease stage and provides insights about the disease that are useful to both the clinical and research community. An example diagnostic according to the present disclosure can provide earlier detection of even small changes in respiration rate which can be indicative of the presence or progression of muscular disabilities, in particular SMA, in a subject and can therefore be used for better disease management including individualized therapy.

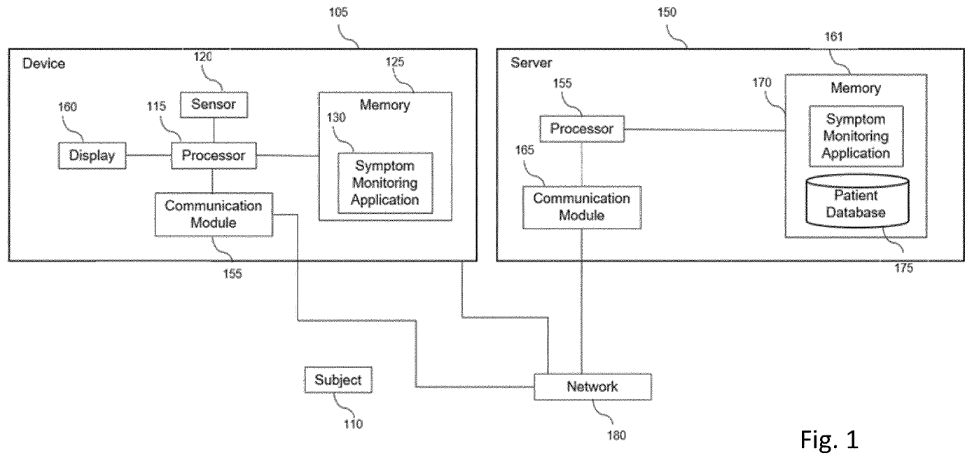

FIG. 1 is a diagram of an example environment in which a diagnostic device 105 for assessing the respiration rate of a muscular disability, in particular SMA, in a subject 110 is provided. In some cases, the device 105 may be a smartphone, a smartwatch or other mobile computing device. The device 105 includes a display screen 160. In some cases, the display screen 160 may be a touchscreen. The device 105 includes at least one processor 115 and a memory 125 storing computer-instructions for a symptom monitoring application 130 that, when executed by the at least one processor 115, cause the device 105 to assess one or more respiration rates of a patient such as a patient with a muscular disability, in particular SMA, and/or to determine an indication of the presence or absence of a muscular disability such as SMA. The device 105 receives a plurality of sensor data via one or more sensors associated with the device 105. In some cases, the one or more sensors associated with the device is at least one of a sensor disposed within the device or a sensor worn by the subject and configured to communicate with the device. In FIG. 1, the sensors associated with the device 105 include a first sensor 120a that is disposed within the display screen 160 of the device 105.

The device 105 extracts, from the received first sensor data a respiration rate.

The device 105 determines the respiration rate of the subject 110 based on the extracted features. In some cases, the device 105 send the extracted features over a network 180 to a server 150. In some cases, the device 105 sends the first sensor data over the network 180 to the server 150. The server 150 includes at least one processor 155 and a memory 161 storing computer-instructions for a symptom assessment application 170 that, when executed by the server processor 155, cause the processor 155 to determine the respiration rate of the subject based on the extracted features received by the server 150 from the device 105. In some cases, the symptom assessment application 170 may determine the respiration rate of the subject 110 based on the extracted features of the sensor data received from the device 105 and a subject database 175 stored in the memory 160. Multiple respiration rate tests may be carried out, with first sensor data collected and processed in each test, such that a plurality of respiration rates may be determined. The symptom assessment application 170 may further determine, from the determined one or more respiration rates, an indication of the presence or absence of a muscular disability such as SMA and may output the indication. In some cases, the subject database 175 may include subject and/or clinical data. In some cases, the subject database 175 may include in-clinic and sensor-based measures of the respiration rate. In some cases, the subject database 175 may be independent of the server 150. In some cases, the server 150 sends the determined one or more respiration rates and/or indication of the presence or absence of the muscular disability to the device 105. In some cases, the device 105 may output the respiration rate and/or the indication. In some cases, the device 105 may communicate information to the subject 110 based on the assessment. In some cases, the assessment of respiration rate or the indication of the presence or absence of the muscular disability, may be communicated to a clinician that may determine individualized therapy for the subject 110 based on the assessment.

In some cases, the computer-instructions for the symptom monitoring application 130, when executed by the at least one processor 115, cause the device 105 to determine the respiration rate of the subject 110 based on active testing of the subject 110. The device 105 prompts the subject 110 to perform one or more tasks. A respiration rate may be calculated for each task. In some cases, prompting the subject to perform the one or more diagnostic tasks includes prompting the subject to take several deep breaths, and to tap the touchscreen (or equivalent sensor) at the beginning of each inhalation cycle, i.e. as they begin to breathe in. Prompting the subject to perform the one or more diagnostic tasks may further include prompting the subject to take several normal breaths, and to tap the touchscreen (or equivalent sensor) at the beginning of each inhalation cycle.

In response to the subject 110 performing each of the one or more diagnostic tasks, the diagnostic device 105 receives a plurality of sensor data via the one or more sensors associated with the device 105, the sensor data comprising a series of time stamps corresponding to the times at which a user indicates (via the sensor) when they have taken a breath. The device 105 extracts, from the received sensor data for each diagnostic task the respiration rate. Thus, a plurality of respiration rates may be determined. The symptoms of a muscular disability, in particular SMA in the subject 110 may include a symptom affecting of the respiration rate of the subject 110.

Thus, the device further may further determine, from the one or more respiration rates, the presence or absence of a muscular disability such as SMA, for example by calculating a frequency ratio. The frequency ratio may be a ratio of a respiration rate corresponding to when the user was prompted to breathe deeply to a respiration rate corresponding to when the user was prompted to breathe normally.

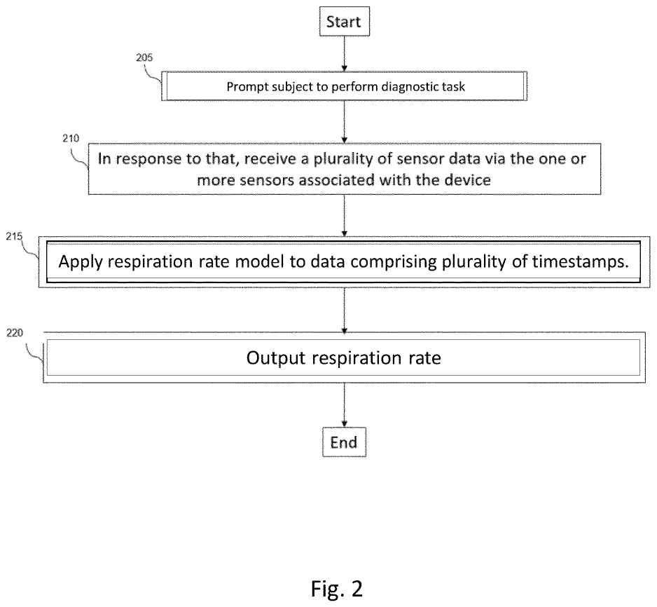

FIG. 2 illustrates an example method for assessing the respiration rate of a subject in a subject based on active testing of the subject using the example device 105 of FIG. 1. While FIG. 2 is described with reference to FIG. 1, it should be noted that the method steps of FIG. 2 may be executed by other systems. The computer-implemented method includes, in step 205, prompting the subject to provide a user input on a user input interface displayed on the display 160 of the device 105 each time the subject is at a predetermined point (such as the beginning) of an inhalation cycle. The method includes receiving, in response to the subject performing the one or more tasks, a plurality of sensor data, via the one or more sensors (step 210), which may be in the form of capacitive sensors in the touchscreen of a display component 160.

Then, in step 215, a respiration rate model is applied to data comprising the plurality of time stamps. The features of the respiration rate model have been explained in detail elsewhere in this patent application, and will not be repeated here, for brevity.

In step 220, a respiration rate is output, e.g. by the processor 115 generating instructions, which when executed by the display component 160 of the device 105 cause the display component 160 to display the respiration rate. Alternatively, the calculated respiration rate may be transmitted to a server 150, as outlined elsewhere in this application.

As discussed above, assessments of symptom severity and progression of a muscular disability, in particular SMA using diagnostics according to the present disclosure correlate sufficiently with the assessments based on clinical results and may thus replace clinical subject monitoring and testing.

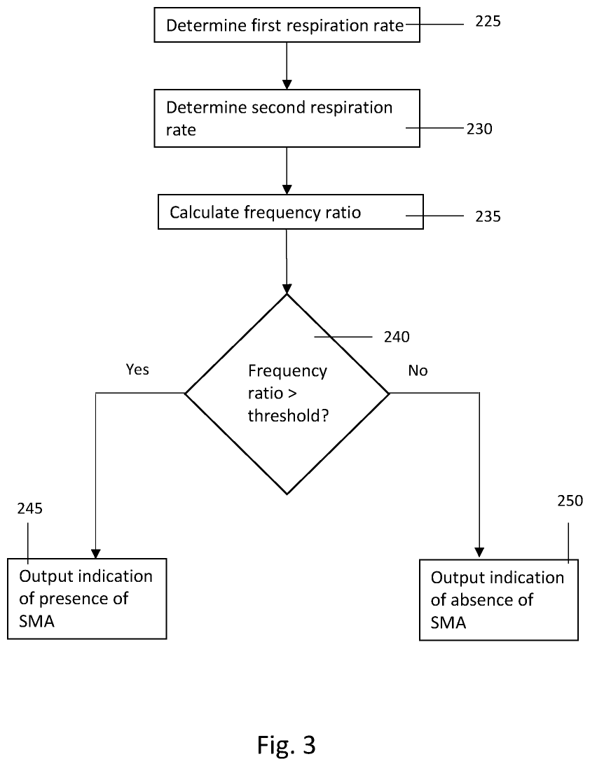

FIG. 3 illustrates an example method for determining an indication of the presence or absence of SMA in a subject based on active testing of the subject using the example device 105 of FIG. 1. While FIG. 3 is described with reference to FIG. 1, it should be noted that the method steps of FIG. 3 may be executed by other systems. The computer-implemented method includes, in steps 225 and 230, determining a first and a second respiration rate, each respiration rate determined according to the method described with reference to FIG. 2. In the test for determining the first respiration rate in step 225, the user may be prompted to breathe normally. In the test for determining the second respiration rate in step 230, the user may be prompted to breathe deeply and slowly. In step 230, the computer-implemented method includes calculating a frequency ratio, which may correspond to the ratio of the second respiration rate to the first respiration rate. Then, in step 240, the computer-implemented method includes determining whether the calculated frequency ratio is greater than a predetermined threshold. If the calculated frequency ratio is determined to be greater than the predetermined threshold, in step 245 the computer-implemented method includes outputting an indication of the presence of SMA. If the calculated frequency ratio is determined to less than or equal to the predetermined threshold, in step 250 the computer-implemented method includes outputting an indication of the absence of SMA.

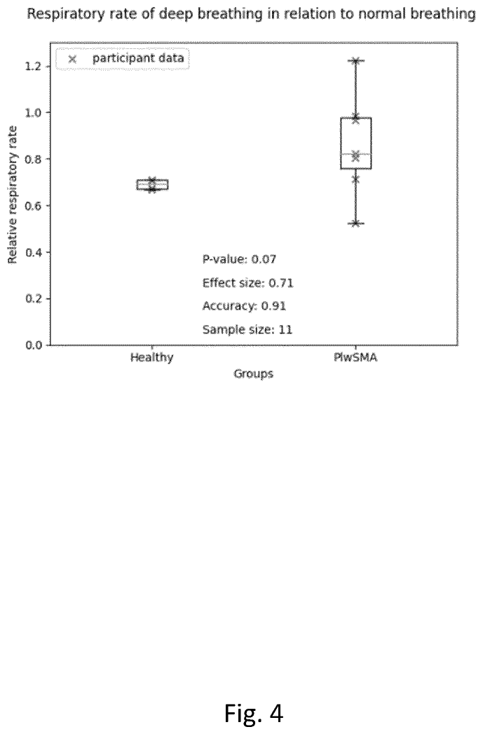

For example, the predetermined threshold may be 0.71. That is, a frequency ratio of >0.71 may indicate that a user is a PlwSMA, and a frequency ratio of <0.71 may indicate that a user is not a PlwSMA. This may be explained with reference to FIG. 4. FIG. 4 is a plot showing calculated frequency ratios for PlwSMA and for healthy individuals. This plot shows that a majority of PlwSMA may have test results of a frequency ratio of >0.71, whereas a majority of healthy individuals may have test results of a frequency ratio of ≤0.71.

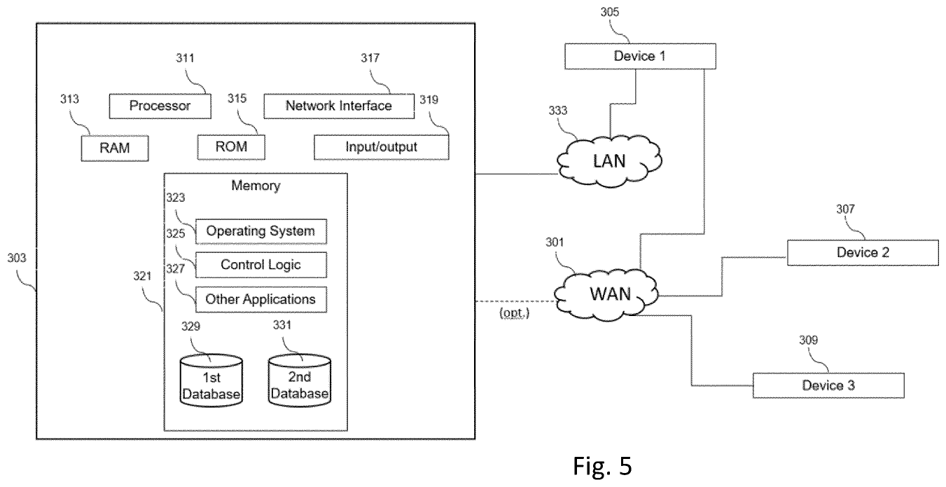

FIG. 5 illustrates an example of a network architecture and data processing device that may be used to implement one or more illustrative aspects described herein, such as the aspects described in FIGS. 1 and 2. Various network nodes 303, 305, 307, and 309 may be interconnected via a wide area network (WAN) 301, such as the Internet. Other networks may also or alternatively be used, including private intranets, corporate networks, LANs, wireless networks, personal networks (PAN), and the like. Network 301 is for illustration purposes and may be replaced with fewer or additional computer networks. A local area network (LAN) may have one or more of any known LAN topology and may use one or more of a variety of different protocols, such as Ethernet. Devices 303, 305, 307, 309 and other devices (not shown) may be connected to one or more of the networks via twisted pair wires, coaxial cable, fibre optics, radio waves or other communication media.

The term “network” as used herein and depicted in the drawings refers not only to systems in which remote storage devices are coupled together via one or more communication paths, but also to stand-alone devices that may be coupled, from time to time, to such systems that have storage capability. Consequently, the term “network” includes not only a “physical network” but also a “content network,” which is comprised of the data-attributable to a single entity which resides across all physical networks.

The components may include data server 303, web server 305, and client computers 307, 309. Data server 303 provides overall access, control and administration of databases and control software for performing one or more illustrative aspects described herein. Data server 303 may be connected to web server 305 through which users interact with and obtain data as requested. Alternatively, data server 303 may act as a web server itself and be directly connected to the Internet. Data server 303 may be connected to web server 305 through the network 301 (e.g., the Internet), via direct or indirect connection, or via some other network. Users may interact with the data server 303 using remote computers 307, 309, e.g., using a web browser to connect to the data server 303 via one or more externally exposed web sites hosted by web server 305. Client computers 307, 309 may be used in concert with data server 303 to access data stored therein, or may be used for other purposes. For example, from client device 307 a user may access web server 305 using an Internet browser, as is known in the art, or by executing a software application that communicates with web server 305 and/or data server 303 over a computer network (such as the Internet). In some cases, the client computer 307 may be a smartphone, smartwatch or other mobile computing device, and may implement a diagnostic device, such as the device 105 shown in FIG. 1. In some cases, the data server 303 may implement a server, such as the server 150 shown in FIG. 1.

Servers and applications may be combined on the same physical machines, and retain separate virtual or logical addresses, or may reside on separate physical machines. FIG. 1 illustrates just one example of a network architecture that may be used, and those of skill in the art will appreciate that the specific network architecture and data processing devices used may vary, and are secondary to the functionality that they provide, as further described herein. For example, services provided by web server 305 and data server 303 may be combined on a single server.

Each component 303, 305, 307, 309 may be any type of known computer, server, or data processing device. Data server 303, e.g., may include a processor 311 controlling overall operation of the rate server 303. Data server 303 may further include RAM 313, ROM 315, network interface 317, input/output interfaces 319 (e.g., keyboard, mouse, display, printer, etc.) , and memory 321. I/O 319 may include a variety of interface units and drives for reading, writing, displaying, and/or printing data or files. Memory 321 may further store operating system software 323 for controlling overall operation of the data processing device 303, control logic 325 for instructing data server 303 to perform aspects described herein, and other application software 327 providing secondary, support, and/or other functionality which may or may not be used in conjunction with other aspects described herein. The control logic may also be referred to herein as the data server software 325. Functionality of the data server software may refer to operations or decisions made automatically based on rules coded into the control logic, made manually by a user providing input into the system, and/or a combination of automatic processing based on user input (e.g., queries, data updates, etc.).

Memory 321 may also store data used in performance of one or more aspects described herein, including a first database 329 and a second database 331. In some cases, the first database may include the second database (e.g., as a separate table, report, etc.). That is, the information can be stored in a single database, or separated into different logical, virtual, or physical databases, depending on system design. Devices 305, 307, 309 may have similar or different architecture as described with respect to device 303. Those of skill in the art will appreciate that the functionality of data processing device 303 (or device 305, 307, 309) as described herein may be spread across multiple data processing devices, for example, to distribute processing load across multiple computers, to segregate transactions based on geographic location, user access level, quality of service (QoS), etc.

One or more aspects described herein may be embodied in computer-usable or readable data and/or computer-executable instructions, such as in one or more program modules, executed by one or more computers or other devices as described herein. Generally, program modules include routines, programs, objects, components, data structures, etc. that perform particular tasks or implement particular abstract data types when executed by a processor in a computer or other device. The modules may be written in a source code programming language that is subsequently compiled for execution, or may be written in a scripting language such as (but not limited to) HTML or XML. The computer executable instructions may be stored on a computer-readable medium such as a hard disk, optical disk, removable storage media, solid state memory, RAM, etc. As will be appreciated by one of skill in the art, the functionality of the program modules may be combined or distributed as desired in various embodiments. In addition, the functionality may be embodied in whole or in part in firmware or hardware equivalents such as integrated circuits, field programmable gate arrays (FPGA), and the like. Particular data structures may be used to more effectively implement one or more aspects, and such data structures are contemplated within the scope of computer executable instructions and computer-usable data described herein.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. A diagnostic device configured to measure a respiration rate of a user, the device comprising:
at least one processor;
a user interface;
one or more sensors associated with the device; and
a memory storing computer-readable instructions that, when executed by the at least one processor, cause the diagnostic device to conduct a respiration rate test, the respiration rate test causing the diagnostic device to:
prompt, via the user interface, the user to provide a user input via the one or more sensors associated with the device each time the user is at a predetermined point during an inhalation cycle;
receive a plurality of user inputs via the one or more sensors, each user input corresponding to a respective time at which the user is at a predetermined point during an inhalation cycle;
generate, in response to receiving each user input, a timestamp associated with the respective user input;
apply a respiration rate model to data comprising the plurality of generated time stamps, wherein the respiration rate model calculates a respiration rate of the user based on the generated time stamps; and
output the calculated respiration rate.

2. A diagnostic device according to claim 1, wherein:
the diagnostic device comprises a smartphone comprising a display component configured to display the user interface.

3. The device of claim 2 wherein:
the display component comprises a touch screen comprising the one or more sensors, and wherein the user input is a screen touch detectable by the one or more sensors.

4. The device of claim 3, wherein the one or more sensors comprise capacitive sensors.

5. The diagnostic device of claim 1 wherein:
the respiration rate model is further configured to calculate a time difference between two time stamps in the plurality of time stamps, and to calculate the respiration rate based on the reciprocal of the time difference.

6. The device of claim 5 wherein:
the earlier of the two time stamps immediately precedes the later of the two time stamps.

7. The device of claim 1, wherein:
the respiration model is further configured to: calculate a plurality of time differences, each time difference being a time difference between two consecutive time stamps; calculate a mean time difference by summing the plurality of time differences and dividing the sum by n or (n−1), where n is the number of time differences; calculate the respiration rate by taking the inverse of the mean time difference.

8. The device of claim 1, wherein:
the unit of the time difference is seconds; and
the respiration rate model further is further configured to multiply the inverted time difference by 60 to obtain the respiration rate in units of breaths per minute.

9. The device of claim 1 wherein:
the predetermined point during the inhalation cycle is the point at which the patient begins to inhale.

10. The device of claim 1 wherein the computer-readable instructions, when executed by the at least one processor, causes the diagnostic device to conduct two respiration rate tests, wherein the user is prompted to breathe at a different breathing rate and/or depth in each respiration rate test.

11. The device of claim 10 wherein the computer-readable instructions, when executed by the at least one processor, causes the diagnostic device to apply a clinical interpretation model to the calculated respiration rates, wherein the clinical interpretation model outputs an indication of the presence or absence of a muscular disability.

12. The device of claim 11, wherein the clinical interpretation model is configured to calculate a frequency ratio of the calculated respiration rates and to determine the indication of the presence or absence of the muscular disability based on the calculated frequency ratio.

13. The device of claim 12, wherein the two respiration rates include a first respiration rate and a second respiration rate, and wherein the frequency ratio corresponds to a ratio of the second respiration rate to the first respiration rate, wherein the first respiration rate corresponds to a respiration rate test in which the user is prompted to breathe normally and the second respiration rate test corresponds to a test in which the user is prompted to breath deeply.

14. The device of claim 13, wherein the clinical interpretation model is configured to compare the frequency ratio to a predetermined value and, based on this comparison, to output the indication of the presence or absence of the muscular disability.

15. The device of claim 14, wherein the clinical interpretation model is configured to determine whether the frequency ratio is greater than a predetermined threshold, and if it is determined that the frequency ratio is greater than the predetermined threshold, to output an indication of the presence of the muscular disability, and if it is determined that the frequency ratio is less than or equal to the predetermined threshold, to output an indication of the absence of the muscular disability.

16. The device of claim 15, wherein the predetermined threshold is at least 0.6, and no greater than 0.8.

17. A computer-implemented method of measuring respiration rate in a patient, the method comprising:
prompting, via a user interface, the subject to provide an input, via one or more sensors, each time the subject is at a predetermined point during an inhalation cycle;
receiving a plurality of user inputs via the one or more sensors;
generating, in response to receiving each user input, a timestamp associated with the respective user input;
applying a respiration rate model to data comprising the plurality of generated time stamps, wherein the respiration rate model is configured to calculate a respiration rate of the user based on the generated timestamps; and
outputting the calculated respiration rate.

18. A computer-implemented method according to claim 17, wherein the computer-implemented method further comprises the steps of:
applying a clinical interpretation model to the calculated respiration rate, wherein the clinical interpretation model outputs an indication of the presence or absence of a muscular disability, or an indication of the progression of a muscular disability.

19. (canceled)

20. A computer-implemented method according to claim 17, wherein the steps of prompting the subject, receiving the user inputs and generating the time-stamps are carried out by a processor of a diagnostic device, and wherein the step of applying the respiration rate model model is carried out by a processor of a server, wherein the diagnostic device is configured to transmit the generated time stamps to the server, and wherein the diagnostic device comprises:
at least one processor;
a user interface;
one or more sensors associated with the device; and
a memory storing computer-readable instructions that, when executed by the at least one processor, cause the diagnostic device to conduct a respiration rate test, the respiration rate test causing the diagnostic device to:
prompt, via the user interface, the user to provide a user input via the one or more sensors associated with the device each time the user is at a predetermined point during an inhalation cycle;
receive a plurality of user inputs via the one or more sensors, each user input corresponding to a respective time at which the user is at a predetermined point during an inhalation cycle;
generate, in response to receiving each user input, a timestamp associated with the respective user input.
